(19) United States
(12) Patent Application Publication
(10) Pub. No.: US 2026/0015569 A1
(43) Pub. Date: Jan. 15, 2026

(54) DETECTION DEVICE

(71) Applicant: Japan Display Inc., Tokyo (JP)

(72) Inventors: Kaoru ITO, Tokyo (JP); Akihiko FUJISAWA, Tokyo (JP); Daichi ABE, Tokyo (JP); Norio MAMBA, Tokyo (JP)

(21) Appl. No.: 19/262,782

(22) Filed: Jul. 8, 2025

(30) Foreign Application Priority Data
Jul. 12, 2024 (JP) 2024-112661

(51) Int. Cl. C12M 1/34; G01N 21/88

(52) U.S. Cl. CPC C12M 41/36; G01N 21/8806

(57) ABSTRACT

A detection device includes: a sensor panel with optical sensors; a light source; a member; and a control circuit. An object to be detected between the sensor panel and the light source is provided with a culture medium. The control circuit repeats an acquisition process to acquire outputs of the optical sensors and performs a comparison process to compare a first output obtained in the first acquisition process with a second output obtained in the latest acquisition process. The comparison process includes: a first comparison process to compare the output for each optical sensor and a second comparison process to compare the output for each area that includes one optical sensor and other optical sensors around the one optical sensor. The control circuit determines the outputs of the optical sensors that satisfy first and second conditions to be the outputs of the optical sensors overlapping the colony.

### CROSS-REFERENCE TO RELATED APPLICATION

This application claims the benefit of priority from Japanese Patent Application No. 2024-112661 filed on Jul. 12, 2024, the entire contents of which are incorporated herein by reference.

### BACKGROUND

### 1. Technical Field

What is disclosed herein relates to a detection device.

### 2. Description of the Related Art

Detection devices are known that enable detection of states of culture environments for culturing biological tissues or microorganisms using an optical sensor (for example, Japanese Patent Application Laid-open Publication No. 2005-087005 (JP-A-2005-087005)).

Sensing of the culture environments by a detection device such as the one in JP-A-2005-087005 is based on the fact that the brightness of light detected by the optical sensor tends to decrease as the culture of the objects to be cultured progresses. In this case, foreign matter such as dust may enter a Petri dish provided with a culture medium (e.g., agar) in which the objects to be cultured are cultured. If the foreign matter moves while the culture progresses, a decrease in brightness of light caused by shadow cast by the foreign matter may be confused with an increase in the objects to be cultured due to the progression of the culture. Such confusion can be a factor decreasing the accuracy of sensing during the progression of the culture of the objects to be cultured. Therefore, there has been a demand for a mechanism that can further reduce or prevent the confusion of the movement of the foreign matter with the increase in number of the objects to be cultured, so as not to decrease the accuracy of sensing during the progression of the culture of the objects to be cultured.

For the foregoing reasons, there is a need for a detection device that can reduce or prevent decrease in the accuracy of sensing.

### SUMMARY

According to an aspect, a detection device includes: a sensor panel that has a detection area in which a plurality of optical sensors are two-dimensionally arranged; a light source configured to emit light; a member on which an object to be detected is to be placed so that the object to be detected is interposed between the detection area and the light source; and a control circuit configured to control operations of the sensor panel and the light source and perform processing based on outputs of the optical sensors. The object to be detected is provided with a culture medium capable of culturing a colony. The control circuit is configured to: perform an acquisition process to operate the light source to generate light traveling toward the sensor panel after the placement of the object to be detected, and acquire the outputs of the sensor panel corresponding to an intensity of the light detected by the optical sensors; repeat the acquisition process at intervals of a predetermined waiting time; and perform a comparison process to compare a first output that is the outputs of the sensor panel obtained in the first acquisition process with a second output that is the outputs of the sensor panel obtained in the latest acquisition process. The comparison process includes: a first comparison process to compare the first output with the second output for each output from each of the optical sensors; and a second comparison process to compare the first output with the second output for each output from an area that includes one optical sensor and other optical sensors located around the one optical sensor. The control circuit is configured to determine the outputs of the optical sensors that satisfy a first condition and a second condition to be the outputs of the optical sensors that overlap the colony. The first condition is that a difference between the first output and the second output is a predetermined difference or larger in the first comparison process. The second condition is that, when the optical sensor determined to satisfy the first condition is set as the one optical sensor, the difference between the first output and the second output is the predetermined difference or larger in the second comparison process.

### BRIEF DESCRIPTION OF THE DRAWINGS

FIG. 1 is a diagram illustrating a main configuration of a detection device;

FIG. 2 is a diagram illustrating a configuration example of a detection area and a wiring area;

FIG. 3 is a circuit diagram illustrating a circuit configuration of an optical sensor;

FIG. 4 is a schematic diagram schematically illustrating a configuration example of a detection system;

FIG. 5 is a schematic diagram illustrating a relation between one detection device and an external configuration;

FIG. 6 is a schematic view illustrating a positional relation between the main configuration of the detection device and an object to be detected;

FIG. 7 is a schematic view illustrating an object irradiated with light from a sensor panel in plan view;

FIG. 8 is a schematic diagram illustrating an outline of a method for detecting a colony;

FIG. 9 is a schematic diagram illustrating a basic mechanism for distinguishing a differential area caused by foreign matter from a differential area caused by expansion of a colony;

FIG. 10 is a schematic diagram illustrating a more developed mechanism for distinguishing the differential area caused by the foreign matter from the differential area caused by the expansion of the colony;

FIG. 11 is a diagram illustrating weighting coefficients in a first unit area and a second unit area when a plurality of pixels are all equivalently weighted;

FIG. 12 is a diagram illustrating examples of the weighting coefficients when the pixels included in the first unit area and the second unit area are individually weighted;

FIG. 13 is a flowchart of processing performed in the detection device when values of M and N in Expression (2) are fixed;

FIG. 14 is a flowchart of processing performed in the detection device when the values of M and N in Expression (2) are varied; and

FIG. 15 is a schematic diagram illustrating a configuration example of a light source 22.

### DETAILED DESCRIPTION

The following describes an embodiment of the present disclosure with reference to the drawings. What is disclosed herein is merely an example, and the present disclosure naturally encompasses appropriate modifications easily conceivable by those skilled in the art while maintaining the gist of the present invention. To further clarify the description, the drawings may schematically illustrate, for example, widths, thicknesses, and shapes of various parts as compared with actual aspects thereof. However, they are merely examples, and interpretation of the present disclosure is not limited thereto. The same element as that illustrated in a drawing that has already been discussed is denoted by the same reference numeral through the description and the drawings, and detailed description thereof may not be repeated where appropriate.

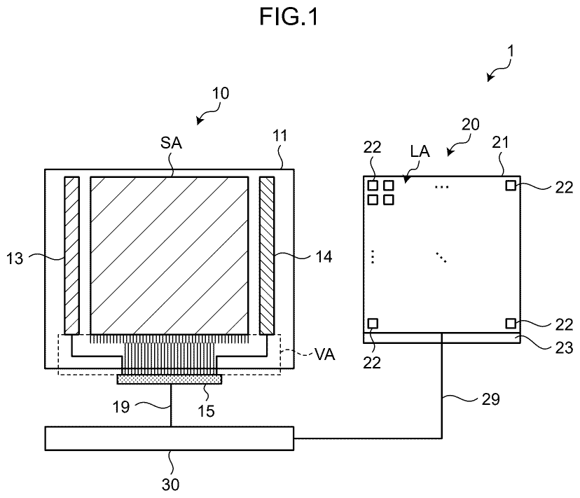

FIG. 1 is a diagram illustrating a main configuration of a detection device 1. The detection device 1 includes a sensor panel 10, a light source panel 20, and a control circuit 30. The sensor panel 10 and the light source panel 20 of the detection device 1 are coupled to the control circuit 30.

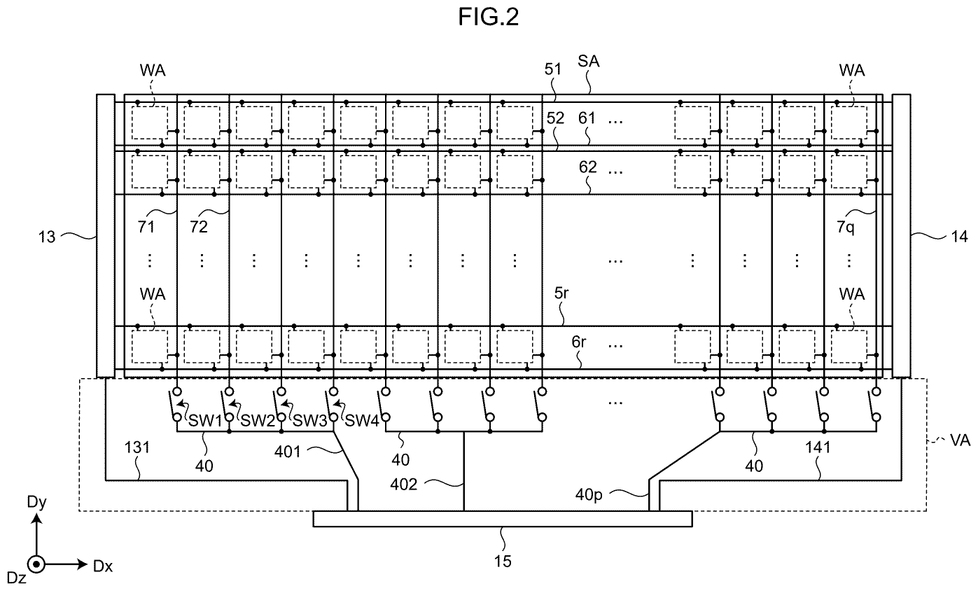

The sensor panel 10 is provided with a detection area SA (refer to FIG. 2) on a substrate 11. A reset circuit 13, a scan circuit 14, and a wiring area VA are mounted on the substrate 11. Components on the detection area SA, the reset circuit 13, and the scan circuit 14 are coupled to a detection circuit 15 via the wiring area VA.

The light source panel 20 has a light-emitting area LA that emits light to the detection area SA. The light source panel 20 is provided with a light source 22 on a substrate 21. The light source 22 includes a light-emitting element such as a light-emitting diode (LED), and is provided in the light-emitting area LA. In the example illustrated in FIG. 1, a plurality of the light sources 22 are arranged in a matrix having a row-column configuration on the substrate 21.

The light source panel 20 is provided with a light source drive circuit 23. Under the control of the control circuit 30, the light source drive circuit 23 controls turning on and off each of the light sources 22, and the luminance thereof when being turned on. The light sources 22 may be provided so as to be individually controllable in light emission or may be provided so as to emit light all together.

The control circuit 30 performs various types of control related to the operation of the detection device 1. Specifically, the control circuit 30 is a circuit, such as a field-programmable gate array (FPGA) or an application-specific integrated circuit (ASIC) that can implement a plurality of functions. The control circuit 30 is coupled to the detection circuit 15 via wiring 19 and acquires outputs from the detection circuit 15. The control circuit 30 is coupled to the light source drive circuit 23 via wiring 29 and performs processing related to the lighting of the light sources 22, such as determination of lighting patterns of the light sources 22.

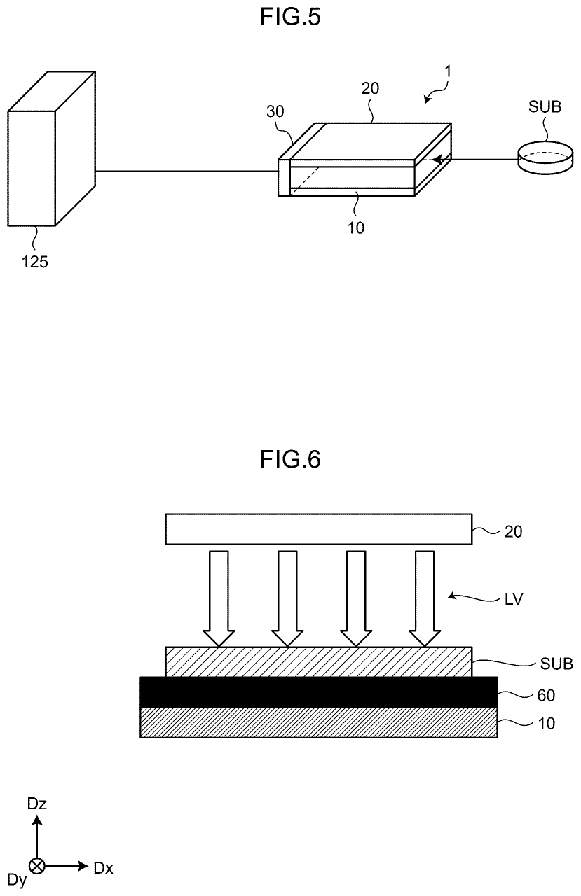

The control circuit 30 also performs processing related to detection of a colony in an object to be detected SUB (refer to FIG. 5). The processing will be described later.

Although not illustrated in the drawings, the detection device 1 includes an analog-to-digital conversion circuit, a digital-to-analog conversion circuit, and other components. The analog-to-digital conversion circuit allows an output from an optical sensor WA (refer to FIG. 2) transmitted through the detection circuit 15 to be handled by arithmetic processing by the control circuit 30. The digital-to-analog conversion circuit makes digital signals generated by the arithmetic processing of the control circuit 30 usable for controlling operations of the sensor panel 10 and the light source panel 20. Each of these circuits may be included, for example, in part or in whole in the control circuit 30, may be a function performed by a circuit mounted on a flexible printed circuit (FPC) provided as the wiring 19 or the wiring 29, or may be implemented in other ways in the detection device 1.

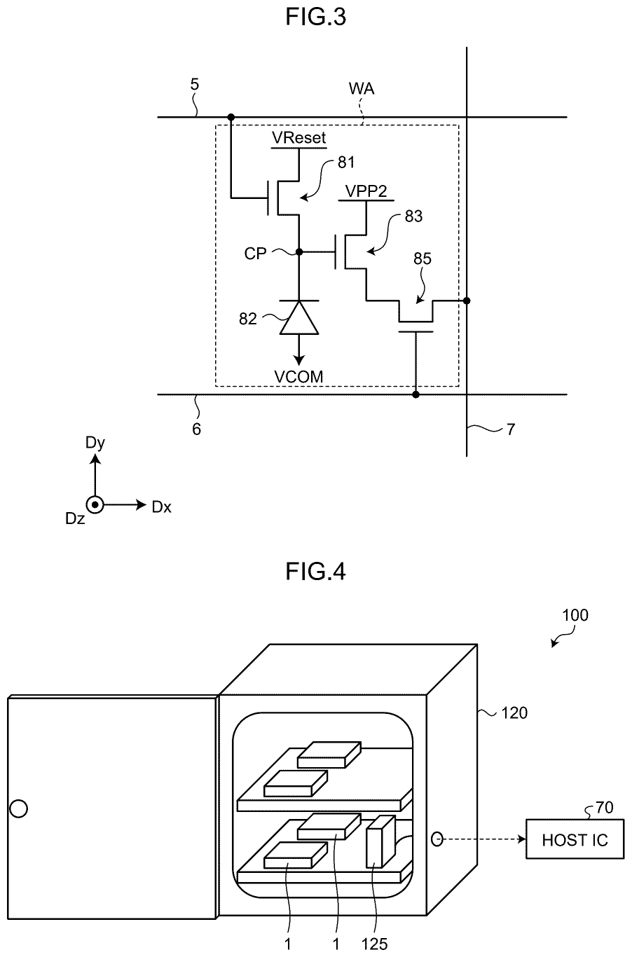

FIG. 2 is a diagram illustrating a configuration example of the detection area SA and the wiring area VA. A plurality of the optical sensors WA (FIG. 3) are provided in the detection area SA. In the embodiment, as illustrated in FIG. 2, the optical sensors WA are arranged in a matrix having a row-column configuration along a first direction Dx and a second direction Dy. The first direction Dx is orthogonal to the second direction Dy. In the following description, the term “third direction Dz” refers to a direction orthogonal to the first direction Dx and the second direction Dy.

The reset circuit 13 is coupled to reset signal transmission lines 51, 52, . . . , 5r. Hereinafter, the term “reset signal transmission line 5” refers to any one of the reset signal transmission lines 51, 52, . . . , 5r. The reset signal transmission line 5 is wiring along the first direction Dx. In the example illustrated in FIG. 2, r reset signal transmission lines 5 are arranged in the second direction Dy. r is a natural number equal to or larger than 2. The r reset signal transmission lines 5 are each coupled, at one end in the first direction Dx, to the reset circuit 13.

The scan circuit 14 is coupled to scan lines 61, 62, . . . , 6r. Hereinafter, the term “scan line 6” refers to any one of the scan lines 61, 62, . . . , 6r. The scan line 6 is wiring along the first direction Dx. In the example illustrated in FIG. 2, r scan lines 6 are arranged in the second direction Dy. The r scan lines 6 are each coupled, at the other end in the first direction Dx, to the scan circuit 14.

As illustrated in FIG. 2, the reset signal transmission lines 5 and the scan lines 6 are alternately arranged in the second direction Dy in the detection area SA. The reset circuit 13 and the scan circuit 14 illustrated in FIGS. 1 and 2 are arranged at locations facing each other with the detection area SA interposed therebetween, but the layout of the reset circuit 13 and the scan circuit 14 is not limited to this layout and can be changed as appropriate.

Signal lines 71, 72, . . . , 7q are also provided in the detection area SA. Hereinafter, the term “signal line 7” refers to any one of the signal lines 71, 72, . . . , 7q. The signal line 7 is wiring along the second direction Dy.

In the example illustrated in FIG. 2, q signal lines 7 are arranged in the first direction Dx. q is a natural number equal to or larger than 2. The q signal lines 7 are each coupled, at one end in the second direction Dy, to one of a plurality of switches (for example, switch SW1, SW2, SW3, or SW4) included in a multiplexer 40.

The multiplexer 40 is provided in the wiring area VA. The multiplexer 40 includes a plurality of switches. In the example illustrated in FIG. 2, the switches SW1, SW2, SW3, and SW4 are illustrated as the switches. The switches included in one multiplexer 40 are turned on (conducting state) at different times from one another. During a period when one of the switches included in one multiplexer 40 is on (conducting state), the other switches are off (non-conducting state). The number of the multiplexers 40 depends on the number (q) of the signal lines 7. When the number of the switches is p, q/p is sufficient as the number of the multiplexers 40. When more than one multiplexer 40 are provided, each of the multiplexers 40 is coupled to the detection circuit 15 via an individual one of wiring lines 401, 402, . . . , 40p.

The coupling between the signal lines 7 and the detection circuit 15 via the multiplexer 40 is merely exemplary and is not limited to this example. The signal lines 7 may be individually directly coupled to the detection circuit 15 in the wiring area VA. In the wiring area VA, the reset circuit 13 is coupled to the detection circuit 15 via wiring 131. In the wiring area VA, the scan circuit 14 is coupled to the detection circuit 15 via wiring 141.

In the detection of light by a PD 82 (refer to FIG. 3) provided in the optical sensor WA, the detection circuit 15 controls the operation timing of the reset circuit 13 and the scan circuit 14. The detection circuit 15 receives the output from the optical sensor WA. The detection circuit 15 converts signals received from the optical sensors WA into data that can be interpreted by the control circuit 30 and outputs the data to the control circuit 30. The detection circuit 15 of the embodiment is a micro-controller unit (MCU).

FIG. 3 is a circuit diagram illustrating a circuit configuration of the optical sensor WA. The first direction Dx and the second direction Dy in FIG. 3 merely correspond to the directions of the reset signal transmission lines 5, the scan lines 6, and the signal lines 7, and do not exactly indicate the relative positional relation of the circuit configuration in the optical sensor WA.

As illustrated in FIG. 3, a switching element 81, the PD 82, a transistor element 83, and a switching element 85 are provided in the optical sensor WA. The PD 82 is a photodiode (PD). The switching elements 81 and 85 and the transistor element are metal-oxide semiconductor field-effect transistors (MOSFETs).

The gate of the switching element 81 is coupled to the reset signal transmission line 5. One of the source and the drain of the switching element 81 is provided with a reset potential VReset. The other of the source and the drain of the switching element 81 is coupled to the cathode of the PD 82 and the gate of transistor element 83. Hereinafter, the term “coupling part CP” refers to a point where the other of the source and the drain of the switching element 81 is coupled to the cathode of the PD 82 and the gate of transistor element 83. A reference potential VCOM is provided from the anode side of the PD 82. The potential difference between the reset potential VReset and the reference potential VCOM is set in advance, but the reset potential VReset and the reference potential VCOM may be variable. The reset potential VReset is higher than the reference potential VCOM.

The drain of the transistor element 83 serving as a source follower is provided with an output source potential VPP2. The source of the transistor element 83 is coupled to one of the source and the drain of the switching element 85. The other of the source and the drain of the switching element 85 is coupled to the signal line 7. The gate of the switching element 85 is coupled to the scan line 6.

The reset potential VReset, the reference potential VCOM, and the output source potential VPP2 are supplied by the detection circuit 15 to the optical sensor WA based on, for example, electric power supplied via a power supply circuit (not illustrated) coupled to the detection circuit 15, but are not limited to being supplied in this way, and may be supplied in a different way as appropriate.

The output source potential VPP2 is set in advance. The potential on the source side of the transistor element 83 is a potential lower than the output potential of the PD 82 by a voltage (Vth) between the gate and the source of the transistor element 83. In this case, the potential on the source side of the transistor element 83 depends on the reset potential VReset and the reference potential VCOM. The potential of the output of the PD 82 corresponds to the photovoltaic power generated by the PD 82 and corresponding to the light detected by the PD 82 during an exposure period.

When the gate of the switching element 85 is turned on by a signal provided from the scan circuit 14 via the scan line 6, the source and the drain of the switching element 85 are brought into a conducting state therebetween. This operation transmits, to the signal line 7 via the switching element 85, a signal (potential) transmitted via the transistor element 83 to the switching element 85. Thus, the output from the optical sensor WA is generated. Hereinafter, the term “scan signal” refers to the signal (potential) provided from the scan circuit 14 via the scan line 6. The scan circuit 14 is a circuit that outputs the scan signal.

The output of one PD 82 provided in one optical sensor WA corresponds to the intensity of the light detected by the PD 82 during the exposure period set in advance. The output of the PD 82 is reset in response to a signal provided by the reset circuit 13 via the reset signal transmission line 5. When the signal turns on the gate of the switching element 81, the source and the drain of the switching element 81 are brought into a conducting state therebetween. This operation resets the potential of the coupling part CP to the reset potential VReset.

FIG. 4 is a schematic diagram schematically illustrating a configuration example of a detection system 100 including the detection device 1. As illustrated in FIG. 4, the detection system 100 includes a plurality of the detection devices 1, a host integrated circuit (IC) 70, and a coupling circuit 125. The detection devices 1 are electrically coupled to the common host IC 70 via the coupling circuit 125.

An incubator 120 illustrated in FIG. 4 is maintained such that an environment (temperature, humidity, and the like) therein is suitable for culturing colonies at the object to be detected SUB while a door is closed. The detection devices 1 are placed in the incubator 120. The object to be detected SUB is provided with a culture medium (e.g., agar) in which the colony can be cultured.

FIG. 5 is a schematic diagram illustrating a relation between one of the detection devices 1 and an external configuration. As illustrated in FIG. 5, the detection device 1 is coupled to the coupling circuit 125 by coupling the control circuit 30 to the coupling circuit 125. As illustrated in FIG. 5, the sensor panel 10 faces the light source panel 20. A gap where the object to be detected SUB can be placed is provided between the sensor panel 10 and the light source panel 20.

The object to be detected SUB is formed of a light-transmitting member and has the culture medium formed on the upper side thereof. The culture medium is a medium capable of culturing the colony. The term simply called “colony” refers to a colony formed by biological tissues or microorganisms cultured in the culture medium formed on the object to be detected SUB. More specifically, the object to be detected SUB is, for example, a glass Petri dish, but is not limited thereto, and may have another configuration that functions in the same way. The culture medium formed on the object to be detected SUB does not have a totally light-blocking property and has such a degree of light-transmitting property that the degree of light transmission varies depending on the presence or absence of the colony and the thickness of the colony.

FIG. 6 is a schematic view illustrating a positional relation between the main configuration of the detection device 1 and the object to be detected SUB. When placing the object to be detected SUB between the sensor panel 10 and the light source panel 20, the object to be detected SUB is placed on a member 60, as illustrated in FIG. 6, for example. The member 60 serves as a member on which the object to be detected SUB can be placed so that the object to be detected SUB is interposed between the detection area SA and the light source panel 20.

In the embodiment, the sensor panel 10 is positioned below the object to be detected SUB and the light source panel 20 is positioned above the object to be detected, as illustrated in FIGS. 5 and 6. The member 60 of the embodiment also serves as an optical member that limits the light emitted from the light sources 22 of the light source panel 20 and reaching the sensor panel 10. Specifically, the member 60 includes any one of a plate-shaped louver, a cylindrical opening, and a microlens. The plate-shaped louver has a plurality of plate-like structures arranged in parallel and having plate surfaces along the third direction Dz. The structures are preferably made of a material having a strong light-absorbing property. The member 60 is provided along a plane (Dx-Dy plane) orthogonal to the third direction Dz. The cylindrical opening penetrates the member 60 in the third direction Dz with respect to the base of the member 60. The base is preferably made of a material having a strong light-absorbing property. The microlens is a small lens with an optical axis along the third direction Dz. The base of the member 60 that supports the microlens is preferably made of a material having a strong light-absorbing property. Whether the member 60 includes the plate-shaped louver, the cylindrical opening, or the microlens, the member 60 as the optical member is provided in order to limit the traveling direction of the light emitted from the light sources 22 and reaching the sensor panel 10 to the third direction Dz or to a direction having a shallower inclination angle with respect to the third direction Dz.

In the embodiment, the member 60 serves as both the optical member and the member on which the object to be detected SUB can be placed. However, the member on which the object to be detected SUB can be placed may be provided separately from the optical member. For example, the member on which the object to be detected SUB can be placed may be a plate-like member provided with a hole capable of accommodating therein the object to be detected SUB. The arrangement of the light source panel 20 and the sensor panel 10 may be reversed. In that case, the member 60 is arranged, for example, above the object to be detected SUB and between the object to be detected SUB and the sensor panel 10.

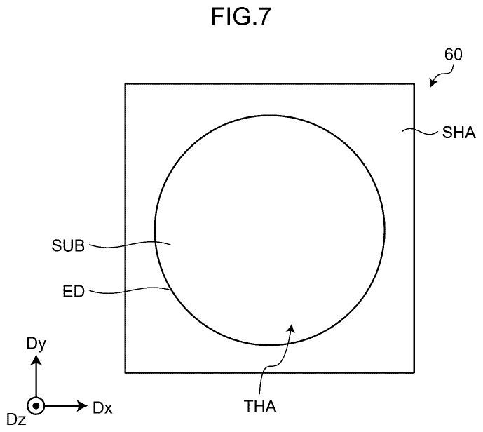

FIG. 7 is a schematic view illustrating an object irradiated with light from the sensor panel 10 in plan view. The term “plan view” refers to a view when a plane along the first direction Dx and the second direction Dy is viewed face-to-face. An area THA is an area on one surface of the member 60 facing the object to be detected SUB and overlaps the object to be detected SUB. An area SHA is an area on the one surface of the member 60 facing the object to be detected SUB and does not overlap the object to be detected SUB. A boundary ED is a boundary between the area THA and the area SHA.

An intensity pattern indicating intensities of light detected by the optical sensors WA two-dimensionally arranged along the Dx-Dy plane indicates degrees of transmission of light through the area THA and the area SHA in the detection area SA. Assuming an output corresponding to the intensity of light detected by one optical sensor WA as a gradation value of one pixel, the combination of the outputs of the optical sensors WA arranged in the detection area SA can be regarded as a two-dimensional image by a combination of a plurality of pixels. In the following description, the term “image” refers to a two-dimensional image generated by the control circuit 30 by combining the outputs of the respective optical sensors WA arranged in the detection area SA, unless otherwise noted. In the following description, the term “scan process” refers to a process in which the control circuit 30 operates the light sources 22 of the light source panel 20 to generate the light traveling toward the sensor panel 10, and the control circuit 30 acquires the outputs of the sensor panel 10 corresponding to the intensities of the light detected by the optical sensors WA arranged in the detection area SA to generate the image.

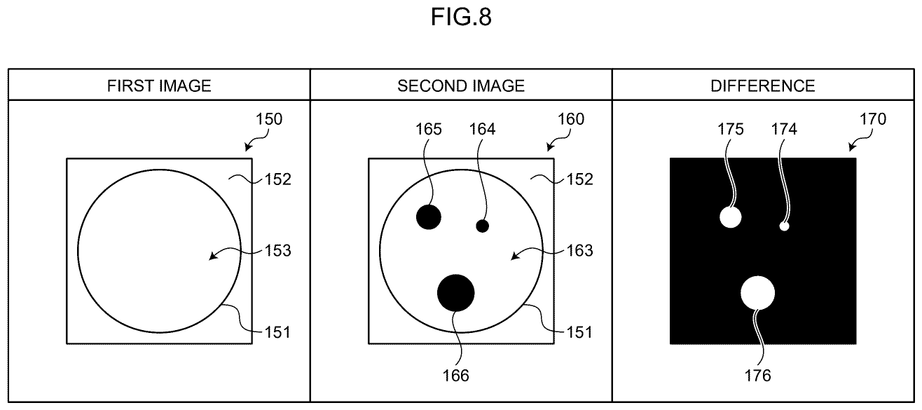

The above describes the configuration serving as a prerequisite for the detection of light by the optical sensors WA provided in the detection area SA, with reference to FIGS. 1 to 7. The following describes a method for detecting the colony using the image, with reference to FIGS. 8 to 14.

FIG. 8 is a schematic diagram illustrating an outline of the method for detecting the colony. “First image” in FIG. 8 is an image before the colony grows. “Second image” in FIG. 8 is an image after the colony has grown. An image 150 illustrated in the “first image” in FIG. 8 includes a boundary 151, an outside portion 152, and an inside portion 153. The outside portion 152 is a portion on the outer side of the boundary and corresponds to a portion outside the object to be detected SUB (or the culture medium). The inside portion 153 is a portion on the inner side of the boundary and corresponds to a portion inside the object to be detected SUB (or the culture medium). An image 160 illustrated in the “second image” in FIG. 8 includes the boundary 151, the outside portion 152, and an inside portion 163. The inside portion 163 is a portion on the inner side of the boundary and corresponds to a portion inside the object to be detected SUB (or the culture medium). The inside portion 163 includes dark parts 164, 165, 166. The growth of the colony occurs because microorganisms or the like are sufficiently cultured in the culture medium.

The boundary 151 is a dark part of the image 160 that is generated correspondingly to the boundary ED as brightness/darkness (variations of light and shade). Specifically, the boundary 151 is formed by outputs corresponding to intensities of light detected by the optical sensors WA arranged overlapping the boundary between the area SHA and the area THA in plan view. The outside portion 152 is a portion of the image 160 that is generated corresponding to intensities of light transmitted through the area SHA as brightness/darkness (variations of light and shade). Specifically, the outside portion 152 is formed by outputs corresponding to the intensities of the light detected by the optical sensors WA arranged overlapping the area SHA in plan view.

The inside portion 153 and the inside portion 163 are portions of the image 160 that is generated corresponding to intensities of light transmitted through the area THA as brightness/darkness (variations of light and shade). Specifically, the inside portion 153 is formed by outputs corresponding to the intensities of the light detected by the optical sensors WA arranged overlapping the area THA in plan view. The inside portion 153 indicates the intensities of the light transmitted through the area THA before the colony grows. The inside portion 163 indicates the intensities of the light transmitted through the area THA after the colony has grown. The inside portion 163 includes the dark parts 164, 165, 166 generated by the growth of the colony, while the inside portion 153 does not include the dark parts 164, 165, 166. That is, the development and growth of the colony causes a decrease in degree of detection of the light in the area where the colony has developed and grown. That is, the dark parts 164, 165, and 166 are each generated by the growth of the colony in the object to be detected SUB overlapping the area THA in plan view. The area where the colony has grown has relatively lower light transmittance than that of the culture medium itself provided in the object to be detected SUB. As a result, the dark parts 164, 165, 166 in the image 160 are relatively darker than the other parts of the inside portion 163. The intensities of the light detected in the inside portion 153 reflect the light-transmitting property of the culture medium formed in the object to be detected SUB and the light-transmitting property of a light-transmitting material forming the object to be detected SUB. The intensities of the light in portions other than the dark parts 164, 165, 166 in the inside portion 163 can also be described in the same manner as in the case of the inside portion 153.

The boundary 151 and the outside portion 152 are the same between the image 150 and the image 160. This is because the appearance of the relatively darker parts, such as the dark parts 164, 165, and 166, due to the growth of the colony is limited to an area corresponding to the inside of the area THA where the colony can grow in the object to be detected SUB. The boundary 151 and the outside portion 152 do not overlap the inside of the area THA. Therefore, the boundary 151 and the outside portion 152 are formed in the image 150 and the image 160 with the output corresponding to substantially the same light intensity, regardless of whether before or after the growth of the colony.

“Difference” in FIG. 8 is the difference between “first image” and “second image”. A differential image 170 illustrated in “difference” in FIG. 8 includes differential areas 174, 175, and 176. The differential area 174 corresponds to the dark part 164 in the image 160. The differential area 175 corresponds to the dark part 165 in the image 160. The differential area 176 corresponds to the dark part 166 in the image 160.

The inside portion 153 of the image 150 does not include the dark parts 164, 165, and 166. In contrast, the inside portion 163 of the image 160 includes the dark parts 164, 165, and 166. Therefore, the differential image 170 as “difference” between the image 150 as the “first image” and the image 160 as the “second image” illustrated in FIG. 8 includes the differential areas 174, 175, and 176 corresponding to the dark parts 164, 165, and 166, as differences between the inside portion 153 and the inside portion 163. When the total area of the differential areas (e.g., the differential areas 174, 175, and 176) in the “difference” (e.g., the differential image 170) is equal to or larger than a predetermined area (size), the control circuit 30 determines that the colony has sufficiently grown in the culture medium in the object to be detected SUB.

In the embodiment, immediately after the object to be detected SUB is placed in the detection device 1 (refer to FIG. 5), the scan process is performed to acquire an image (such as the image 150) as illustrated in the “first image” in FIG. 8 as an initial image. Then, the scan process is performed again each time a predetermined waiting time elapses. After the elapse of the predetermined waiting time occurs more than once, the control circuit 30 performs the process to obtain the difference between the image acquired in the latest scan process and the initial image. This operation obtains the difference (such as the differential image 170) as illustrated in the “difference” in FIG. 8. Thus, if the image acquired in the latest scan process is the image as illustrated in the “second image” (such as the image 160), the differential image 170 is acquired as the difference.

The predetermined waiting time is five minutes, for example, but is not limited thereto, and can be changed as appropriate. The predetermined waiting time is preferably appropriately set according to conditions, such as a growth rate of the colony that is assumed based on environmental conditions in the incubator 120 accommodating the detection device 1 holding therein the object to be detected SUB. Hereinafter, the term “comparison process” refers to the process to obtain the difference between the image acquired in the latest scan process and the initial image.

In this way, the control circuit 30 operates the light sources 22 to generate the light traveling toward the sensor panel 10 after the placement of the object to be detected SUB, and performs acquisition processes to acquire the outputs of the sensor panel 10 corresponding to the light intensities detected by the optical sensors WA. The first of these acquisition processes is an acquisition process to acquire the initial image. An acquisition process performed later than the first acquisition process is the acquisition process to acquire the “image acquired in the latest scan process”. Acquisition processes performed later than the first acquisition process are repeated at intervals of the predetermined waiting time.

The comparison process is a process to compare a first output that is the outputs of the sensor panel 10 obtained in the first acquisition process with a second output that is the outputs of the sensor panel 10 obtained in the latest acquisition process. In this case, the initial image corresponds to the first output. The image acquired in the latest scan process corresponds to the second output.

The appearance of the darker parts obtained by comparing the inside portion 153 with the inside portion 163, such as the dark parts 164, 165, and 166, is not limited to the result of the growth of the colony. For example, unintended foreign matter (e.g., dust) entering the object to be detected SUB also causes such dark parts to appear. Hereinafter, the term simply called “foreign matter” refers to foreign matter such as dust that is already mixed in the object to be detected SUB when the object to be detected SUB is placed in the detection device 1, unless otherwise noted. The dark parts caused by the foreign matter occur from the time of the initial image, that is, the “first image” in FIG. 8. Therefore, even if the foreign matter is mixed in the object to be detected SUB, the foreign matter does not appear as a difference between the “first image” and the “second image” in the “difference” such as the differential image 170, unless the foreign matter moves in the object to be detected SUB. In other words, if the foreign matter moves in the object to be detected SUB, the foreign matter causes a difference between the “first image” and the “second image” in the “difference”.

In the embodiment, a determination process is performed based on the ratio of the number of pixels included in the differential areas to the number of pixels included in the image. The determination process is a process to determine whether the area of the differential areas, such as the differential areas 174, 175, and 176, in the differential image 170 has become equal to or larger than the predetermined area. In the comparison process performed before such a determination process, pixel-by-pixel comparison is performed between the initial image and the image acquired in the latest scan process. As a result of the pixel-by-pixel comparison, pixels having different gradation values (light intensities detected by the optical sensors WA) are regarded as pixels in the differential area. Therefore, when the foreign matter moves in the object to be detected SUB, the differential areas are formed by both pixels corresponding to the outputs of the optical sensors WA overlapping the position of the foreign matter before the movement in plan view and pixels corresponding to the outputs of the optical sensors WA overlapping the position of the foreign matter after the movement in plan view. Such differential areas caused by the foreign matter can cause erroneous determination in the determination process for detecting the growth of the colony.

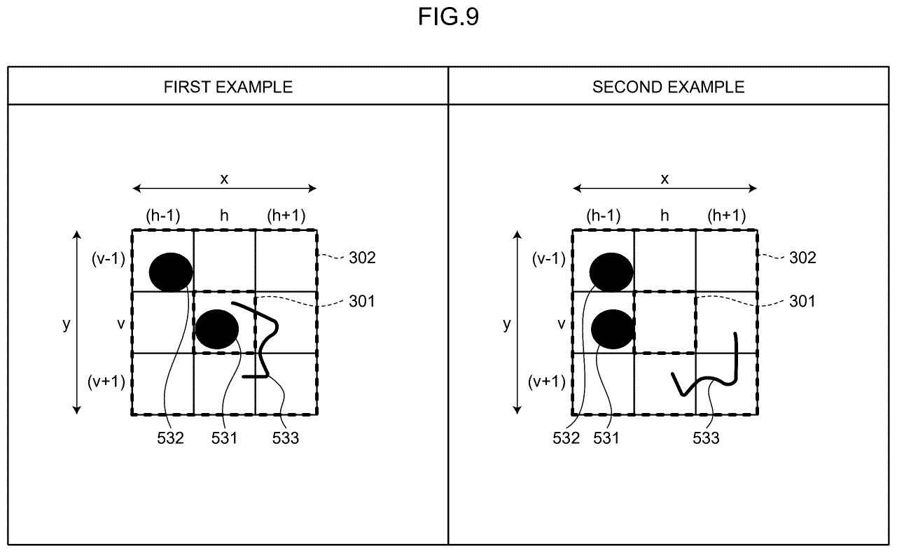

Therefore, in the embodiment, a mechanism is provided to distinguish between the differential areas caused by the foreign matter and the differential areas caused by the growth of the colony. Therefore, in the embodiment, even if the foreign matter mixed in the object to be detected SUB has moved within the object to be detected SUB, it is possible to reduce or prevent misidentification of the differential areas caused by the foreign matter as the growth of the colony. The following describes such a mechanism with reference to FIGS. 9 to 14.

FIG. 9 is a schematic diagram illustrating a basic mechanism for distinguishing the differential area caused by the foreign matter from the differential area caused by the growth of the colony. FIG. 9 illustrates 3×3 pixels of q×r pixels included in the image. Coordinates (x, y)=(h, v) are assigned to a pixel located at the center of the 3×3 pixels. Furthermore, coordinates of the pixels included in an area (first unit area) including the 3×3 pixels are indicated by a combination of (h−1), h, and (h+1) in an x direction and (v−1), v, and (v+1) in a y direction. The x direction in the image corresponds to the arrangement of the optical sensors WA in the first direction Dx in the detection area SA. The y direction in the image corresponds to the arrangement of the optical sensors WA in the second direction Dy in the detection area SA.

“First example” in FIG. 9 illustrates the first unit area included in the initial image and positions of foreign matter 531, 532, and 533 in the first unit area. “Second example” in FIG. 9 illustrates the first unit area included in the image acquired in the scan process later than the scan process to acquire the initial image, and the positions of the foreign matter 531, 532, and 533 in the first unit area. The position of each piece of the foreign matter 531, 532, and 533 changes between the “first example” and the “second example”. In particular, in the “first example”, the foreign matter 531 is in a position overlapping the pixel at (x, y)=(h, v), while in the “second example”, the foreign matter 531 is in a position overlapping the pixel at (x, y)=(h−1, v).

As described above, in the comparison process, pixel-by-pixel comparison is performed between the initial image and the image acquired in the latest scan process. As a result of the pixel-by-pixel comparison, the pixels having different gradation values (light intensities detected by the optical sensors WA) are regarded as the pixels in the differential area. More specifically, a pixel corresponding to I_{d}(x, y) that satisfies Expression (1) below is regarded as a pixel in the differential area.

I_{d}(x,y)=|I(x,y)−I_{b}(x,y)| (1)

I(x, y) in Expression (1) is the gradation value of a pixel in the image acquired in the latest scan process. I_{b}(x, y) in Expression (1) is the gradation value of a pixel in the initial image. Thus, I_{d}(x, y) in Expression (1) is the absolute value of the difference between the gradation value of the pixel in the image acquired in the latest scan process and the gradation value of the pixel in the initial image. The coordinates of the pixel of interest in I(x, y), I_{b}(x, y), and I_{d}(x, y) in Expression (1) are the same and specified by values of (x, y). For example, in the comparison process for the pixel at (x, y)=(h, v), (x, y)=(h, v) is substituted for each of I(x, y), I_{b}(x, y), and I_{d}(x, y).

In the embodiment, a pixel for which I_{d}(x, y)=0 is not satisfied is regarded as a pixel in the differential area. Therefore, the pixel at (x, y)=(h, v) and the pixel at (x, y)=(h−1, v) illustrated in FIG. 9 are regarded as pixels in the differential area when the image in the “second example” is acquired. However, the pixel at (x, y)=(h, v) and the pixel at (x, y)=(h−1, v) are regarded as pixels in the differential area because the foreign matter 531 has moved. Since the position of the foreign matter 533 is also different between the “first example” and the “second example”, the pixels at (x, y)=(h+1, v), (x, y)=(h, v+1), and (x, y)=(h+1, v+1) may also be regarded as pixels in the differential area. Thus, when the pixels in the differential area is determined based only on the value of I_{d}(x, y) calculated by Expression (1), it is difficult to reduce or prevent the appearance of the pixels in the differential area due to changes in positions of foreign matter such as the foreign matter 531 and 533.

Therefore, in the embodiment, a filter process is performed. In the filter process in the embodiment, processing is performed to obtain an overall difference of a plurality of pixels included in a predefined filter process unit area. More specifically, pixels corresponding to I_{D}(x, y) that satisfy Expression (2) below are regarded as pixels in the differential area.

I_{D}(x,y)=|Σ_{n=-N}^{N}Σ_{m=-M}^{M}I(x+m,y+n)−Σ_{n=-N}^{N}Σ_{m=-M}^{M}I_{b}(x+m,y+n)| (2)

I(x+m, y+n) in Expression (2) is the gradation value of the pixel in the image acquired in the latest scan process. I_{b}(x+m, y+n) in Expression (2) is the gradation value of the pixel in the initial image. in Expression (2), m is a variable that is an integer value in the range from −M to M. n is a variable that is an integer value in the range from −N to N. In the example in FIG. 9, M=N=1. Therefore, when the 3×3 pixels that are centered on (x, y)=(h, v) and have coordinate values of ±1 for x and y among the pixels of the image acquired in the latest scan process are referred to as latest unit pixels, and the 3×3 pixels that are centered on (x, y)=(h, v) and have coordinate values of ±1 for x and y among the pixels of the initial image are referred to as initial unit pixels, I_{D}(x, y) in Expression (2) is the absolute value of the difference between the sum of the gradation values of the latest unit pixels and the sum of the gradation values of the initial unit pixels.

In the examples illustrated in FIG. 9, when focused on an area 301 including only the pixel at (x, y)=(h, v), the area 301 in the “first example” differs from the area 301 in the “second example” in terms of presence or absence of the foreign matter 531. Therefore, Id(x, y) obtained by Expression (1) is not zero. In contrast to this, when focused on an area 302 of the 3×3 pixels that are centered on (x, y)=(h, v) and have coordinate values of ±1 for x and y, the area 302 in the “first example” and the area 302 in the “second example” are the same in that the foreign matter 531, 532, and 533 are present in the area 302. Therefore, I_{D}(x, y) obtained by Expression (2) is zero.

Assume that a dark part due to growth of the colony has appeared in the area 301 separately from the foreign matter 531, 532, and 533 at the time of the “second example”. Such a dark part is a new dark part not generated in the “first example”. Therefore, the difference due to such a dark part is reflected in I_{D}(x, y) obtained by Expression (2). That is, I_{D}(x, y) is not zero when the dark part due to the growth of the colony appears.

In the embodiment, when both I_{d}(x, y) obtained by Expression (1) and I_{D}(x, y) obtained by Expression (2) are equal to or higher than a predetermined threshold Th, the pixel at (x, y)=(h, v) is regarded as a pixel in the differential area. That is, in this case, the dark part due to the growth of the colony is considered to have appeared in the pixel at (x, y)=(h, v). In contrast to this, when at least one of I_{d}(x, y) obtained by Expression (1) and I_{D}(x, y) obtained by Expression (2) is lower than the threshold Th, the pixel at (x, y)=(h, v) is not regarded as a pixel in the differential area. That is, in this case, a dark part is regarded to have not appeared in the pixel at (x, y)=(h, v), or even if a dark part has appeared, it is regarded to be due to the movement of the foreign matter. The threshold Th is preferably a value exceeding 0 and is preferably set as appropriate in order to well distinguish a dark part caused by foreign matter from a dark part caused by growth of a colony. As an example, the threshold Th is 10.

The process to obtain I_{d}(x, y) using Expression (1) and the process to obtain I_{D}(x, y) using Expression (2) are individually performed for each of the pixels in the image. Thus, the comparison process includes a first comparison process and a second comparison process. The first comparison process is a process to compare the first output (initial image) with the second output (image acquired in the latest scan process) for each output from each of the optical sensors WA. The process to calculate I_{d}(x, y) using Expression (1) corresponds to the first comparison process. The second comparison process is a process to compare the first output with the second output for each output from an area (such as the area 302, or an area 303 to be described later) that includes one of the optical sensors WA and other optical sensors WA located around the one optical sensor WA. The process to calculate I_{D}(x, y) using Expression (2) corresponds to the second comparison process.

The control circuit 30 determines that, when the output of the optical sensor WA satisfies first and second conditions, the output is an output of the optical sensor WA that has detected light in an area where a dark part is generated due to its overlapping with the colony. The first condition is that the difference between the first output (initial image) and the second output (image acquired in the latest scan process) is a predetermined difference or larger in the first comparison process described above, that is, the process to calculate I_{d}(x, y) using Expression (1). The second condition is that, when the optical sensor WA determined to satisfy the first condition is set as the one of the optical sensors WA (optical sensor WA that has produced the output corresponding to the gradation value of the pixel at (x, y)=(h, v)), the difference between the first output (initial image) and the second output (the image acquired by the latest scan process) is the predetermined difference or greater in the second comparison process, that is, the process to calculate I_{D}(x, y) using Expression (2). The predetermined difference or greater refers to a difference equal to or higher than the threshold Th.

In the example illustrated in FIG. 9, the values of M and N for calculating I_{D}(x, y) in Expression (2) are M=N=1, but the values of M and N are not limited thereto. For example, I_{D}(x, y) may be calculated a plurality of times by varying the values of M and N.

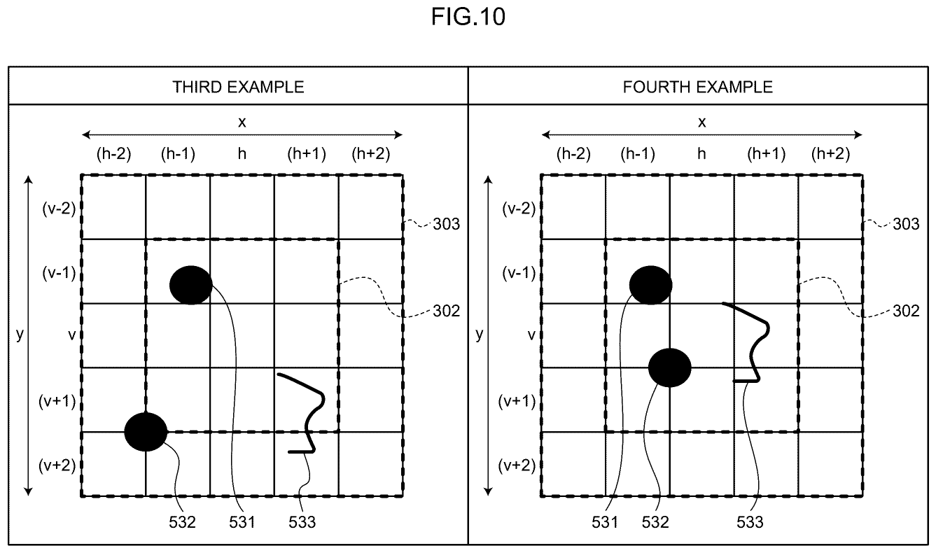

FIG. 10 is a schematic diagram illustrating a more developed mechanism for distinguishing the differential area caused by the foreign matter from the differential area caused by the growth of the colony. FIG. 10 illustrates 5×5 pixels of q×r pixels included in the image. The coordinates (x, y)=(h, v) are assigned to a pixel located at the center of the 5×5 pixels. Furthermore, coordinates of the pixels included in an area (second unit area) including the 5×5 pixels are indicated by a combination of (h−2), (h−1), j, (h+1), and (h+2) in the x direction and (v−2), (v−1), v, (v+1), and (v+2) in the y-direction.

FIG. 10 also illustrates the area 302 and the area 303. The area 301 is the area of the 3×3 pixels that are centered on the pixel at (x, y)=(h, v) and have coordinate values of ±1 for x and y. The area 303 is the area of the 5×5 pixels that are centered on the pixel at (x, y)=(h, v) and have coordinate values of ±2 for x and y. “Third example” in FIG. 10 illustrates the second unit area included in the initial image and the positions of the foreign matter 531, 532, and 533 in the second unit area. “Fourth example” in FIG. 10 illustrates the second unit area included in the image acquired in the scan process later than the scan process to acquire the initial image, and the positions of the foreign matter 531, 532, and 533 in the second unit area.

In the “third example” in FIG. 10, a part of the foreign matter 532 and 533 is located inside the area 302 and other part is located outside the area 302. In contrast to this, in the “fourth example”, the whole of the foreign matter 532 and 533 are located inside the area 302. If the values of M and N in calculating I_{D}(x, y) in Expression (2) are M=N=1, the 3×3 pixels that are subject to calculation of differences in gradation values of the pixels in I_{D}(x, y) are pixels included in the area 302. The differences in gradation values between the area 302 in the “third example” and the area 302 in the “fourth example” are generated by a difference in ratio of the foreign matter 532 and 533 included in the area 302. Therefore, if the values of M and N are M=N=1, I_{D}(x, y) obtained by Expression (2) is not zero.

In contrast to this, if the values of M and N in calculating I_{D}(x, y) using Expression (2) are M=N=2, the 5×5 pixels subject to the calculation of the differences in gradation values of the pixels in I_{D}(x, y) are the pixels included in the area 303. The area 303 in the “third example” and the area 303 in the “fourth example” are the same in that the foreign matter 531, 532, and 533 are located in the area 303. Therefore, if the values of M and N are M=N=2, I_{D}(x, y) obtained by Expression (2) is zero.

As described in the examples with reference to the “third example” and the “fourth example” in FIG. 10, the appearance of the differential area caused by the movement of the foreign matter can be highly accurately reduced or prevented by calculating I_{D}(x, y) not only when M=N=1 but also when M=N=2. Thus, in the second comparison process, that is, the process to calculate I_{D}(x, y) using Expression (2), the first output (initial image) may be compared with the second output (image acquired in the latest scan process) for each output from each of a first area (such as the area 302) and a second area (such as the area 303). The first area and the second area both include pixels corresponding to the optical sensors WA that include one optical sensor WA (optical sensor WA that has produced the output corresponding to the gradation value of the pixel at (x, y)=(h, v)) and the other optical sensors WA arranged around the one optical sensor WA, but the number of the other optical sensors WA is different between the first area and the second area. In this case, the second condition described above may be satisfied when the difference between the first output and the second output is the predetermined difference or larger in both the first area and the second area, and the second condition described above may be unsatisfied when the difference between the first output and the second output is less than the predetermined difference in at least one of the first and second areas.

When calculating I_{D}(x, y) using Expression (2), more than one of the pixels included in the first unit area and the second unit area may be individually weighted or may not be individually weighted. When no such individual weighting is applied, all the pixels that are subject to calculation of differences are equally treated when calculating I_{D}(x, y).

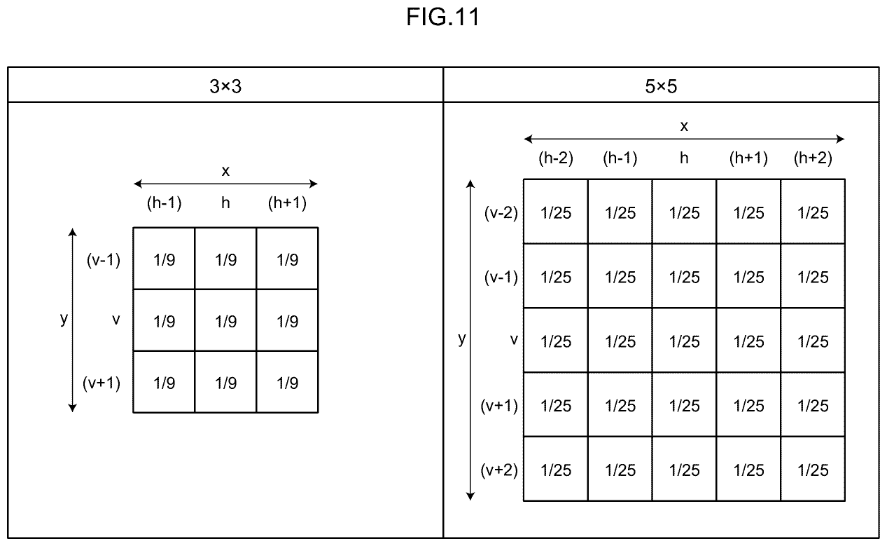

FIG. 11 is a diagram illustrating weighting coefficients in the first unit area and the second unit area when more than one of the pixels are all equivalently weighted. A “3×3” averaging filter illustrated in FIG. 11 indicates that the weighting coefficients applied to the respective 3×3 pixels included in the first unit area are all the same value of “1/9”. When the “3×3” averaging filter illustrated in FIG. 11 is applied, in calculating I_{D}(x, y) using Expression (2) with the setting of M=N=1, the entire right side of Expression (2) is multiplied by 1/9. This calculation is equivalent to multiplying I_{D} (X, y) by 1/9 in the calculation using Expression (2). A “5×5” averaging filter illustrated in FIG. 11 indicates that the weighting coefficients applied to the respective 5×5 pixels included in the second unit area are all the same value of “1/25”. When the “5×5” averaging filter illustrated in FIG. 11 is applied, in calculating I_{D}(x, y) using Expression (2) with the setting of M=N=2, the entire right side of Expression (2) is multiplied by 1/25. This calculation is equivalent to multiplying I_{D}(x, y) by 1/25 in the calculation using Expression (2).

According to the weighting factors illustrated in FIG. 11, a multiplying factor corresponding to the same value of the weighting coefficients is applied to the value of I_{D}(x, y), so that the relative relation with the threshold Th changes. However, the magnitude of effect on the value of I_{D}(x, y) does not greatly differ depending on the position of the pixel in the unit area determined according to the values of M and N. The term “unit area” mentioned above refers to an expression that encompasses the first unit area and the second unit area described above. The same applies hereinafter when the term “unit area” is simply used.

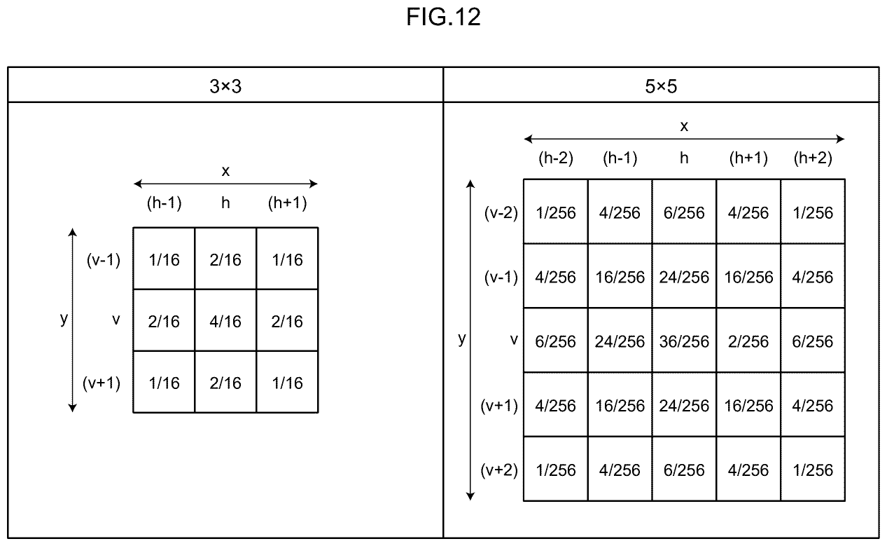

FIG. 12 is a diagram illustrating examples of the weighting coefficients when the pixels included in the first unit area and the second unit area are individually weighted. A “3×3” Gaussian filter illustrated in FIG. 12 indicates that, among the 3×3 pixels included in the first unit area, “4/16” is applied to the pixel located at (x, y)=(h, v); “2/16” is applied to the pixels located at (x, y)=(h+1, v) and (x, y)=(h, v+1); and “1/16” is applied to the pixels located at (x, y)=(h±1, v±1). When the “3×3” Gaussian filter illustrated in FIG. 12 is applied, in calculating I_{D}(x, y) using Expression (2) with the setting of M=N=1, the entire right side of Expression (2) is multiplied by 4/16 when 0 is substituted for m and n in Expression (2). Furthermore, in this case, when 0 is substituted for one of m and n in Expression (2) and ±1 is substituted for the other, the entire right side of Expression (2) is multiplied by 2/16. Moreover, in this case, when ±1 is substituted for m and n in Expression (2), the entire right side of Expression (2) is multiplied by 1/16.

A “5×5” Gaussian filter illustrated in FIG. 12 indicates that, among the 5×5 pixels included in the second unit area, “36/256” applied to the pixel located at (x, y)=(h, v); “24/256” applied to the pixels located at (x, y)=(h+1, v) and (x, y)=(h, v±1); “16/256” is applied to the pixels located at (x, y)=(h±1, v±1); “6/256” is applied to the pixels located at (x, y)=(h, v±2) and (x, y)=(h±2, v); “4/256” is applied to the pixels located at (x, y)=(h±1, v±2) and (x, y)=(h±2, v±1); and “1/256” is applied to the pixels located at (x, y)=(h±2, v±2) and (x, y)=(h±2, v±2). When the “5×5” Gaussian filter illustrated in FIG. 12 is applied, in calculating I_{D}(x, y) using Expression (2) with the setting of M=N=2, the entire right side of Expression (2) is multiplied by 36/256 when 0 is substituted for m and n in Expression (2). Furthermore, in this case, when 0 is substituted for one of m and n in Expression (2) and ±1 is substituted for the other, the entire right side of Expression (2) is multiplied by 24/256. Moreover, in this case, when ±1 is substituted for m and n in Expression (2), the entire right side of Expression (2) is multiplied by 16/256. In addition, in this case, when 0 is substituted for one of m and n in Expression (2) and ±2 is substituted for the other, the entire right side of Expression (2) is multiplied by 6/256. Furthermore, in this case, when ±1 is substituted for one of m or n in Expression (2) and ±2 is substituted for the other, the entire right side is multiplied by 4/256. Moreover, in this case, when ±2 is substituted for m and n in Expression (2), the entire right side of Expression (2) is multiplied by 1/256.

According to the weighting factors illustrated in FIG. 12, the magnitude of the effect on the value of I_{D}(x, y) varies depending on the position of the pixel in the unit area determined according to the values of M and N. Specifically, the differences in the gradation values of the pixels located closer to the center of the unit area affect more greatly the value of I_{D}(x, y). In other words, the differences in the gradation values of the pixels located farther from the center of the unit area have smaller effects on the value of I_{D}(x, y). Therefore, assume, for example, a case where the foreign matter is located in the unit area, and the foreign matter in the initial image is located in a position farther from (x, y)=(h, v), that is, in a position overlapping a pixel near an edge of the unit area. Furthermore, in this case, assume that the position of the foreign matter changes from a position overlapping a pixel near an edge of the unit area in the initial image to a position out of the unit area in the latest image. In this case, the Gaussian filter described above works so as to reduce the degree of effect on the value of I_{D}(x, y) by the change in gradation values of the pixels near the edge of the unit area, compared with the position at (x, y)=(h, v) that is the center of the unit area. Thus, in this case, the Gaussian filter makes the degree of change in the value of I_{D}(x, y) caused by the movement of the foreign matter smaller than the averaging filter does. In contrast to this, assume, for example, a case where the foreign matter is located in the unit area, and the foreign matter in the initial image is located in a position closer to (x, y)=(h, v). Furthermore, in this case, assume that the foreign matter has moved out of the unit area in the latest image. In this case, the Gaussian filter makes the degree of change in the value of I_{D}(x, y) caused by the movement of the foreign matter larger than the averaging filter does.

Thus, the weighting corresponding to the Gaussian filter can cause a degree of change in the value of I_{D}(x, y) to depend on the position in which the foreign matter has been located with respect to (x, y)=(h, v) before moving. In other words, regarding the movement of the foreign matter that has overlapped the pixel near the edge of the unit area in the initial image, the effect on the value of I_{D}(x, y) can be made smaller. As described with reference to FIG. 12, in the second comparison process, that is, the process to calculate I_{D}(x, y) using Expression (2), the weighting may be individually applied to each of the outputs of one optical sensor WA (pixel at (x, y)=(h, v)) and the other optical sensors WA located around the one optical sensor WA. FIG. 12 indicates that the degree of weighting for the output of the one optical sensor WA is higher than the weighting for each of the outputs of the other optical sensors WA. FIG. 12 also indicates that the degree of weighting for the output of a first optical sensor WA of two optical sensors WA is higher than the weighting for the output of a second optical sensor WA of the two optical sensors WA, wherein the two optical sensors WA are included in the other optical sensors WA and adjacent to each other in the detection area SA, and the first optical sensor WA of the two adjacent optical sensors WA is located closer to the above-described one optical sensor WA than the second optical sensor WA of the two adjacent optical sensors WA is.

In the embodiment, the control circuit 30 performs the calculation of I_{d}(x, y) obtained by Expression (1) and I_{D}(x, y) obtained by Expression (2), and the determination of “whether to regard the pixel at (x, y)=(h, v) as a pixel in the differential area” based on I_{d}(x, y) and I_{D}(x, y), but the host IC 70 may perform these calculation and determination.

For example, the host IC 70 performs the determination on the growth of the colony based on the number of the pixels regarded as the differential area, but the control circuit 30 may perform this determination. Specifically, for example, the host IC 70 determines that the colony has sufficiently grown on the object to be detected SUB when the ratio of the number of the pixels regarded as the differential area in the latest image has become a predetermined ratio or higher with respect to the number of the optical sensors WA arranged in the detection area SA of the sensor panel 10 (100%), but the control circuit 30 may perform this determination. The predetermined ratio is 5%, for example, but this is one example. The predetermined ratio is not limited to this value, and may be an appropriate ratio depending on the conditions required by objects to be cultured in the colony.

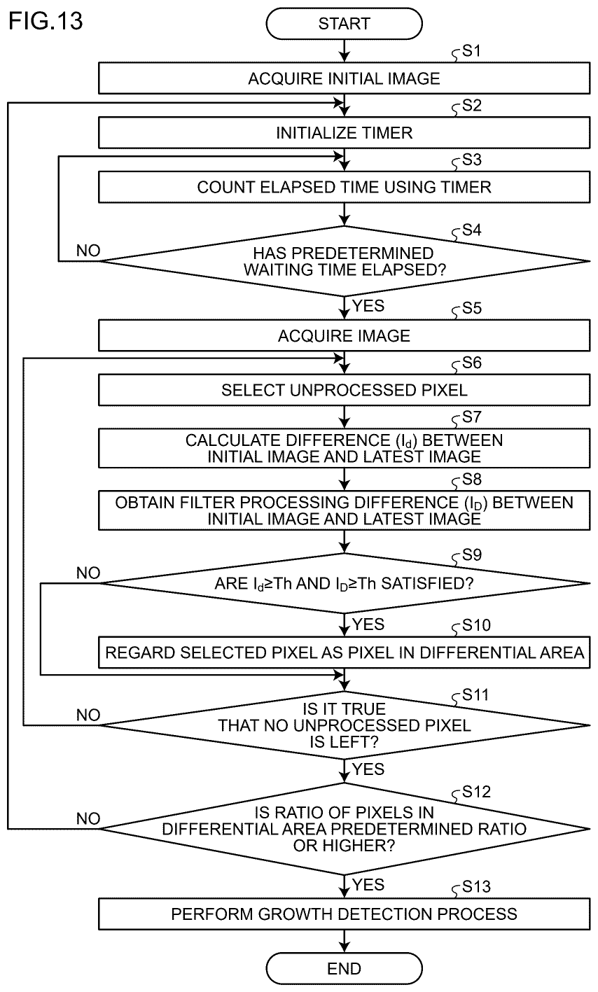

The following describes processing in each of a case where the values of M and N in Expression (2) are fixed (refer to FIG. 9) and a case where the values of M and N in Expression (2) are varied (refer to FIG. 10). First, the processing when the values of M and N in Expression (2) are fixed (refer to FIG. 9) will be described with reference to FIG. 13.

FIG. 13 is a flowchart of processing performed in the detection device 1 when the values of M and N in Expression (2) are fixed. First, the initial image is acquired (Step S1). Specifically, immediately after the object to be detected SUB is placed (refer to FIG. 5) in the detection device 1, the scan process is performed to acquire the image (such as the image 150) as illustrated as the initial image in the “first Image” in FIG. 8. The control circuit 30 generates the image from the outputs of the optical sensors WA obtained in the scan process and holds it as the initial image.

The control circuit 30 then initializes a timer (Step S2). The timer is a timer to count the waiting time before performing again the scan process. The timer is controlled, for example, by a counter variable and a timing clock, but may be controlled by a dedicated timer circuit included in the control circuit 30. After the process at Step S2, the timer counts elapsed time (Step S3). After the process at Step S2, the process at Step S3 is continued until the elapse of the predetermined waiting time has been counted through the process at Step S3 (No at Step S4).

If the predetermined waiting time has elapsed (Yes at Step S4), the image is acquired (Step S5). Specifically, the scan process is performed again. The control circuit 30 generates the image from the outputs of the optical sensors WA obtained in the scan process as the latest image.

After the process at Step S5, one unprocessed pixel is selected from among the pixels of the latest image acquired in the process at Step S5 (Step S6). The term “unprocessed pixel” herein refers to a pixel that has not yet been subjected to processing from Step S7 to Step S9 to be described below.

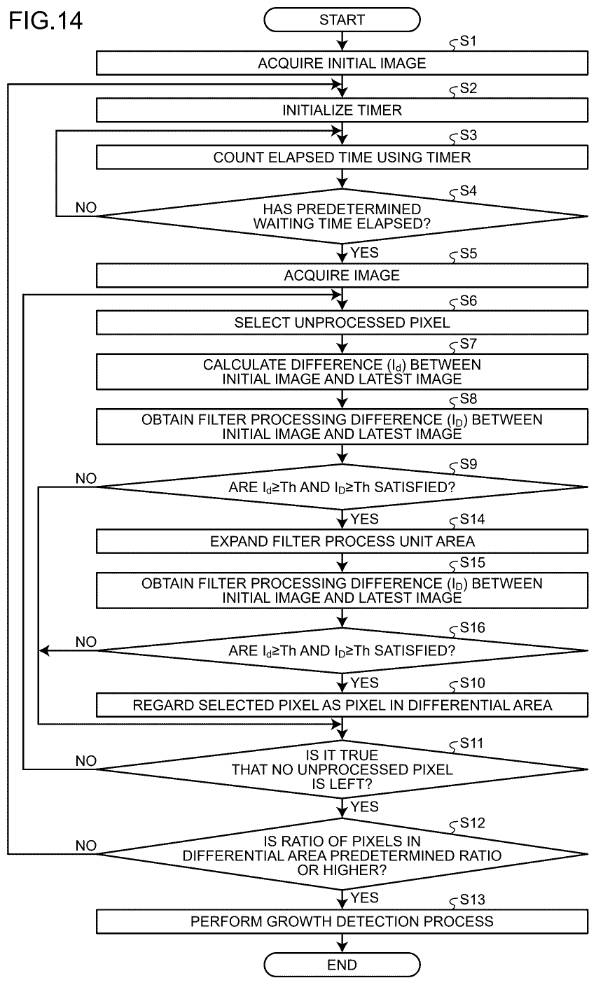

The control circuit 30 calculates I_{d}(x, y) using Expression (1) described above with the coordinates of the pixel selected in the process at Step S6 as (x, y)=(h, v) (Step S7). I_{d}(x, y) can be said to be the value of the difference to which a filter described with reference to FIGS. 13 and 14 is not applied. The control circuit 30 calculates I_{D}(x, y) using Expression (2) described above with the coordinates of the pixel selected in the process at Step S6 as (x, y)=(h, v) (Step S8). I_{D}(x, y) can be said to be the value of the difference (filter processing difference) to which the filter described with reference to FIGS. 13 and 14 is applied. In other words, in the process at Step S8, the weighting corresponding to the Gaussian filter as illustrated in FIG. 14 may be performed, or I_{D}(x, y) may be calculated with substantially no weighting corresponding to the averaging filter as illustrated in FIG. 13. In each of the processes at Step S7 and Step S8, the absolute value indicating the difference between the pixel of the initial image acquired in the process at Step S1 and the pixel of the latest image acquired in the process at Step S5 is calculated.

After the processes at Steps S7 and S8, the control circuit 30 determines whether both I_{d}(x, y) obtained in the process at Step S7 and I_{D}(x, y) obtained in the process at Step S8 are equal to or higher than the threshold Th (Step S9). If both I_{d}(x, y) and I_{D}(x, y) are determined to be equal to or higher than the threshold Th (Yes at Step S9), the control circuit 30 regards the pixel selected in the process at Step S6 as a pixel in the differential area (Step S10). That is, in this case, the dark part due to the growth of the colony is determined to have appeared in the pixel selected in the process of Step S6. After the process at Step S9, the control circuit 30 determines whether it is true that no unprocessed pixel is left in the latest image acquired in the process at Step S5 (Step S11). If any unprocessed pixel is determined to be left (No at Step S11), the process at Step S6 is performed.

In contrast, in the process at Step S11, if no unprocessed pixel is determined to be left (Yes at Step S11), the host IC 70 determines whether the ratio of the number of the pixels regarded as the differential area in the latest image is the predetermined ratio or higher with respect to the number of the optical sensors WA arranged in the sensor panel 10 (100%) (Step S12). If the ratio of the number of the pixels regarded as the differential area is determined to be the predetermined ratio or higher (Yes at Step S12), a growth detection process is performed (Step S13). Specifically, the host IC 70 transmits an electronic message to report that the colony has sufficiently expanded on the object to be detected SUB to a pre-registered contact address of an administrator of the detection system 100. Such an electronic message is electronic mail, for example, but is not limited thereto, and may be a message of another form or a voice signal that serves in the same way. In contrast to this, if, in the process at Step S12, it is determined that the ratio of the number of the pixels regarded as the differential area is lower than the predetermined ratio (No at Step S12), the process at Step S2 is performed. That is, the acquisition of the latest image performed at intervals of the predetermined waiting time is continued until the colony sufficiently expands on the object to be detected SUB.

In the process at Step S9, if at least one of I_{d}(x, y) and I_{D}(x, y) is determined to be lower than the threshold Th (No at Step S9), the process at Step S11 is performed.

That is, in this case, a dark part is regarded to have not appeared in the pixel selected in the process at Step S6, or even if a dark part has appeared, it is regarded to be due to the movement of the foreign matter.

The following describes the processing when the values of M and N in Expression (2) are varied (refer to FIG. 10), with reference to FIG. 14. In the description of the processing with reference to FIG. 14, the same process as that described with reference to FIG. 13 is assigned the same step number and will not be described.

FIG. 14 is a flowchart of processing performed in the detection device 1 when the values of M and N in Expression (2) are varied. Among the processes illustrated in FIG. 14 performed by the detection device 1 when the values of M and N in Expression (2) are varied, the processing from the process at Step S1 to the process at Step S8 is the same as that when the values of M and N in Expression (2) are fixed (refer to FIG. 13).

In the processing performed by the detection device 1 when the values of M and N in Expression (2) are varied, if both I_{d}(x, y) and I_{D}(x, y) are determined to be equal to or higher than the threshold Th in the process at step S9 (Yes at Step S9), the control circuit 30 expands the filter process unit area (Step S14). Specifically, at least one of the values of M and N is changed to a larger value. A process at Step S15 performed after the process at Step S14 is the same as the process at Step S8, but at least one of the values of M and N is set larger than that of the process at Step S8. In the embodiment, M=N=1 is applied in the process at Step S8, and M=N=2 is applied in the process at Step S15, but these values are examples.

After the process at Step S15, whether both I_{d}(x, y) obtained in the process at Step S7 and I_{D}(x, y) obtained in the process at Step S15 are equal to or higher than the threshold Th (Step S16). If both I_{d}(x, y) and I_{D}(x, y) are determined to be equal to or higher than the threshold Th (Yes at Step S16), the process at Step S10 is performed. In contrast to this, if at least one of I_{d}(x, y) and I_{D}(x, y) is determined to be lower than the threshold Th (No at Step S16), the process at Step S11 is performed. Unless otherwise noted above, the processing described with reference to FIG. 14 is the same as the processing described with reference to FIG. 13.

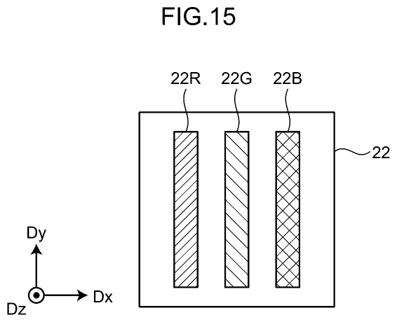

The following exemplifies a configuration common to the embodiment described above and various modifications thereof, with reference to FIG. 15. FIG. 15 is a schematic diagram illustrating a configuration example of the light source 22. As illustrated in FIG. 15, the light source 22 includes a first light source 22R, a second light source 22G, and a third light source 22B. The first light source 22R, the second light source 22G, and the third light source 22B are light-emitting elements (such as LEDs) that emit light in different colors. In the embodiment, the first light source 22R emits red (R) light. The second light source 22G emits green (G) light. The third light source 22B emits blue (B) light.

In the embodiment, the first light source 22R, the second light source 22G, and the third light source 22B are individually controlled to emit light. That is, when one of the first light source 22R, the second light source 22G, and the third light source 22B is turned on, the other two are not turned on. In the detection of the colony described with reference to FIGS. 8 to 14, detection with the first light source 22R turned on (first light detection), detection with the second light source 22G turned on (second light detection), and detection with the third light source 22B turned on (third light detection) are individually performed. If it is determined that the colony has been sufficiently cultured in at least one of the first light detection, the second light detection, and the third light detection, the growth detection process mentioned above is performed. White light may be emitted by simultaneously turning on the first light source 22R, the second light source 22G, and the third light source 22B. In other words, colonies may be detected under conditions of white light irradiation.

As described above, according to the embodiment, the detection device 1 includes the sensor panel (such as the sensor panel 10) that has the detection area SA in which the optical sensors (such as the optical sensors WA) are two-dimensionally arranged, the light sources (such as the light sources 22) that emit light, the member (such as the member 60) on which the object to be detected (such as the object to be detected SUB) can be placed so that the object to be detected is interposed between the detection area SA and the light sources, and the control circuit (such as the control circuit 30) that controls operations of the sensor panel and the light sources and performs the processing based on the outputs of the optical sensors. The object to be detected is provided with the culture medium in which the colony can be cultured. The control circuit performs the acquisition process to operate the light sources to generate light traveling toward the sensor panel after the placement of the object to be detected, and acquire the outputs of the sensor panel corresponding to the intensity of the light detected by the optical sensors. The control circuit repeats the acquisition process at intervals of the predetermined waiting time, and performs the comparison process to compare the first output (such as the initial image) that is the outputs of the sensor panel obtained in the first acquisition process with the second output (such as the image acquired in the latest scan process) that is the outputs of the sensor panel obtained in the latest acquisition process. The comparison process includes the first comparison process (such as the process to calculate I_{d}(x, y) using Expression (1)) to compare the first output with the second output for each output from each of the optical sensors and the second comparison process (such as the process to calculate I_{D}(x, y) using Expression (2)) to compare the first output with the second output for each output from the area that includes the one optical sensor and the other optical sensors located around the one optical sensor. The control circuit determines the outputs of the optical sensors that satisfy the first and the second conditions to be the outputs of the optical sensors that overlap the colony. The first condition is that the difference between the first output and the second output is the predetermined difference or larger in the first comparison process. The second condition is that, when the optical sensor in which the difference between the first output and the second output is determined to be the predetermined difference or larger in the first comparison process is set as the one optical sensor, the difference between the first output and the second output is the predetermined difference or larger in the second comparison process. This method allows the distinction between the movement of the foreign matter (such as the foreign matter 531, 532, and 533) and the increase in the dark parts due to the culture of the colony. Thus, it is possible to further reduce or prevent the confusion between the movement of the foreign matter and the increase in the dark parts due to the culture of the colony and thereby reduce or prevent decrease in the accuracy of sensing during the progression of the culture of the colony.

In the second comparison process to compare the first output (such as the initial image) with the second output (such as the image acquired in the latest scan process) for each output from the area that includes the one optical sensor (such as the optical sensor WA) and the other optical sensors located around the one optical sensor, the first output is compared with the second output for each output from each of the first area (such as the area 302) and the second area (such as the area 303) different in the number of the other optical sensors. The second condition is satisfied when the difference between the first output and the second output is the predetermined difference or larger in both the first and the second areas, and the second condition is not satisfied when the difference between the first output and the second output is less than the predetermined difference in at least one of the first and the second areas, thereby allowing the more accurate distinction between the movement of the foreign matter (such as the foreign matter 531, 532, and 533) and the increase in the dark parts due to the culture of the colony. Therefore, it is possible to further securely reduce or prevent the confusion between the movement of the foreign matter and the increase in the dark parts due to the culture of the colony and thereby reduce or prevent decrease in the accuracy of sensing during the progression of the culture of the colony.

In the second comparison process to compare the first output (such as the initial image) with the second output (such as the image acquired in the latest scan process) for each output from the area that includes the one optical sensor (such as the optical sensor WA) and the other optical sensors located around the one optical sensor, the output of the one optical sensor and the outputs of the other optical sensors are individually weighted. As a result, a portion where the movement of the foreign matter (such as the foreign matter 531, 532, and 533) is to be more weighted can be freely set in the output in the area including the one optical sensor (optical sensor WA) and the other optical sensors located around the one optical sensor.

In the weighting, the weighting for the output of the one optical sensor (such as the optical sensor WA) (such as the pixel at (x, y)=(h, v)) is stronger in degree of weight than the weighting for the output of each of the other optical sensors. The weighting for the output of a first optical sensor of two optical sensors is stronger in degree of weight than weighting for an output of a second optical sensor of the two optical sensors, wherein the two optical sensors are included in the other optical sensors and adjacent to each other in the detection area SA, and the first optical sensor of the two optical sensors is located closer to the one optical sensor than the second optical sensor of two optical sensors is. Therefore, the movement of the foreign matter (such as the foreign matter 531, 532, and 533) that have overlapped the vicinity of an edge of the output of the area including one optical sensor (optical sensor WA) and the other optical sensors located around the one optical sensor in the first output (such as the initial image) can have smaller effects on the second comparison process (such as the process to calculate I_{D}(x, y) using Expression (2)) for the one optical sensor (optical sensor WA).

The optical member (such as the member 60) that limits the light emitted from the light sources (such as the light sources 22) and reaching the sensor panel is provided between the sensor panel (such as the sensor panel 10) and the object to be detected (such as the object to be detected SUB). With this configuration, the light emitted from the light sources and reaching the sensor panel can be easily limited to more preferable light in terms of the detection of the colony on the object to be detected.

The optical member (such as the member 60) includes any one of the plate-shaped louver, the cylindrical opening, and the microlens. With this configuration, the direction of the light emitted from the light sources (such as the light sources 22) and reaching the sensor panel (such as the sensor panel 10) can be more easily limited to a direction in which the light sources face the sensor panel (such as the third direction DZ).

The sensor panel (such as the sensor panel 10) is located below the object to be detected (such as the object to be detected SUB) and the light sources (such as the light sources 22) are located above the object to be detected. With this configuration, the member (such as the member 60) on which the object to be detected can be placed so that the object to be detected is interposed between the detection area SA and the light sources can be more easily made into the same component as the optical member.

The light source 22 illustrated in FIG. 15 has a configuration in which the longitudinal directions of the first light source 22R, the second light source 22G, and the third light source 22B are along the second direction Dy, and the first light source 22R, the second light source 22G, and the third light source 22B are arranged in this order from one side toward the other side in the first direction Dx. This configuration is, however, an exemplary form of the light source 22, which is not limited to this form. For example, the shape of the first light source 22R, the second light source 22G, and the third light source 22B in the light source 22 as viewed in plan view, and the positional relation among the first light source 22R, the second light source 22G, and the third light source 22B can be changed as appropriate. A single white light source may be provided instead of the first light source 22R, the second light source 22G, and the third light source 22B.

The switching elements 81 and 85 illustrated in FIG. 3 are each not limited to the configuration with a single switching element. For example, at least one of the switching element 81 and the switching element 85 may have what is called a double-gate configuration.

The object to be detected, such as the object to be detected SUB, is not limited to the Petri dish in which the culture medium is formed, and may have another configuration. The object to be detected may be, for example, a plate for suspension culture.

The arrangement of the optical sensors WA is not limited to a matrix having a row-column configuration along the first direction Dx and the second direction Dy. For example, the optical sensors WA arranged in the sensor rows adjacent in the second direction Dy need not both be located on a straight line along the second direction Dy. Specifically, the optical sensors WA may be arranged in what is called a staggered manner. From the viewpoint of using a wiring line for both the reset signal transmission line 5 and the scan line 6, the arrangement of the optical sensors WA in the first direction Dx is preferably such that the optical sensors WA are located on a straight line along the first direction Dx, but this arrangement is also not essential and can be changed as appropriate within a range of not impairing the functions of the optical sensors WA and the detection area SA. The arrangement of the light sources 22 in the light source panel 20 is also not limited to a matrix having a row-column configuration and can be any arrangement.

The values of M and N are not limited to 1 or 2. A natural number of three or larger may be applied to the values of M and N. It is not necessary to satisfy M=N. That is, the unit area need not be an area where the number of pixels arranged in the x direction is equal to the number of pixels arranged in the y-direction.

Other operational advantages accruing from the aspects described in the present embodiment that are obvious from the description herein, or that are conceivable as appropriate by those skilled in the art will naturally be understood as accruing from the present disclosure.

What is claimed is:

1. A detection device comprising:
a sensor panel that has a detection area in which a plurality of optical sensors are two-dimensionally arranged;
a light source configured to emit light;
a member on which an object to be detected is to be placed so that the object to be detected is interposed between the detection area and the light source; and
a control circuit configured to control operations of the sensor panel and the light source and perform processing based on outputs of the optical sensors, wherein
the object to be detected is provided with a culture medium capable of culturing a colony,
the control circuit is configured to:
perform an acquisition process to operate the light source to generate light traveling toward the sensor panel after the placement of the object to be detected, and acquire the outputs of the sensor panel corresponding to an intensity of the light detected by the optical sensors;
repeat the acquisition process at intervals of a predetermined waiting time; and
perform a comparison process to compare a first output that is the outputs of the sensor panel obtained in the first acquisition process with a second output that is the outputs of the sensor panel obtained in the latest acquisition process,
the comparison process comprises:
a first comparison process to compare the first output with the second output for each output from each of the optical sensors; and
a second comparison process to compare the first output with the second output for each output from an area that includes one optical sensor and other optical sensors located around the one optical sensor,
the control circuit is configured to determine the outputs of the optical sensors that satisfy a first condition and a second condition to be the outputs of the optical sensors that overlap the colony,
the first condition is that a difference between the first output and the second output is a predetermined difference or larger in the first comparison process, and
the second condition is that, when the optical sensor determined to satisfy the first condition is set as the one optical sensor, the difference between the first output and the second output is the predetermined difference or larger in the second comparison process.

2. The detection device according to claim 1, wherein
in the second comparison process, the first output is compared with the second output for each output from each of a first area and a second area different in the number of the other optical sensors, and
the second condition is satisfied when the difference between the first output and the second output is the predetermined difference or larger in both the first area and the second area, and the second condition is not satisfied when the difference is less than the predetermined difference in at least one of the first area and the second area.

3. The detection device according to claim 1, wherein the output of the one optical sensor and the outputs of the other optical sensors are individually weighted in the second comparison process.

4. The detection device according to claim 3, wherein
weighting for the output of the one optical sensor is stronger in degree of weight than weighting for the output of each of the other optical sensors, and
weighting for an output of a first optical sensor of two optical sensors is stronger in degree of weight than weighting for an output of a second optical sensor of the two optical sensors, wherein the two optical sensors are included in the other optical sensors and adjacent to each other in the detection area, and the first optical sensor of the two optical sensors is located closer to the one optical sensor than the second optical sensor of the two optical sensors is.

5. The detection device according to claim 1, wherein an optical member configured to limit the light emitted from the light source and reaching the sensor panel is provided between the sensor panel and the object to be detected.

6. The detection device according to claim 5, wherein the optical member comprises any one of a plate-shaped louver, a cylindrical opening, and a microlens.

7. The detection device according to claim 1, wherein
the sensor panel is located below the object to be detected, and
the light source is located above the object to be detected.
